(19) United States
(12) Patent Application Publication
(10) Pub. No.: US 2023/0293237 A1
(43) Pub. Date: Sep. 21, 2023

(54) SURGICAL SYSTEMS, METHODS, AND DEVICES EMPLOYING AUGMENTED REALITY (AR) INSTRUMENT GUIDANCE

(71) Applicant: Depuy Synthes Products, Inc., Raynham, MA (US)

(72) Inventors: Alexander Samaha, Boston, MA (US); Nicolas Demanget, Saint-Egréve (FR); Cory Emil, Milton, MA (US); Roman Lomeli, Plymouth, MA (US)

(21) Appl. No.: 18/122,771

(22) Filed: Mar. 17, 2023

Related U.S. Application Data
(60) Provisional application No. 63/321,618, filed on Mar. 18, 2022.

(51) Int. Cl. A61B 34/10; A61B 34/20; A61B 90/00

(52) U.S. Cl. CPC A61B 34/10; A61B 34/20; A61B 90/36; A61B 2034/102; A61B 2034/105; A61B 2034/107; A61B 2034/2046; A61B 2090/365

(57) ABSTRACT

Systems and methods are disclosed for computer aided surgery (CAS), comprising an augmented reality (AR) system configured to display augmented reality information, a position tracking system configured to track positions (e.g., location and orientation) of objects, an instrument coupled to a tracker detectable by the position tracking system, and a controller configured to: display augmented reality information using the AR system, the augmented reality information comprising: an indication of a predetermined (e.g., planned) target plane for a cut on the bone (for example as a plurality of first indicators), and an indication of a cut that would be produced by a current orientation of the instrument (for example as a plurality of second indicators), determine, as the instrument is moved, when the cut that would be produced by the current orientation of the instrument is aligned with the predetermined target plane, and based on the determined alignment, display augmented reality information comprising an indication representing that the current orientation of the instrument is aligned with the predetermined target plane (for example as a plurality of third indicators).

### CROSS REFERENCE TO RELATED APPLICATION

This application claims priority to U.S. Provisional Application Ser. No. 63/321,618, filed Mar. 18, 2022, the contents of which are hereby incorporated by reference in its entirety.

### BACKGROUND

Many surgical procedures require large amounts of information for planning and/or undertaking the procedure. One way to manage this is to improve the way information is presented to a user, e.g., a surgeon. Augmented Reality (AR) provides an overlay of virtual information on or adjacent to a “real-world” object visually perceived by a user, usually through an AR device such as a headset, head mounted device (HMD), Google Glass, etc. The AR device may display information, such as pictures, video, text, warnings, models, simulations, etc., while not obscuring the user's view of the real-world objects in her proximity.

However, the information displayed may be selectable, pertinent, and customizable. For example, intra-op planning can greatly benefit from AR systems, provided it does not negatively impact workflow. Specific use cases, such as navigation relative to a plane, may present challenges that can be at least ameliorated by properly configured AR systems.

Accordingly, there is a need for improved systems, methods, and devices to employ AR that can improve patient outcome and surgical efficiency.

### SUMMARY

Systems and methods are disclosed for computer aided surgery (CAS), comprising an augmented reality (AR) system configured to display augmented reality information, a position tracking system configured to track positions (e.g., location and orientation) of objects, an instrument coupled to a tracker detectable by the position tracking system, and a controller configured to: display augmented reality information using the AR system, the augmented reality information comprising: an indication of a predetermined (e.g., planned) target plane for a cut on the bone, and an indication of a cut that would be produced by a current orientation of the instrument, determine, as the instrument is moved, when the cut that would be produced by the current orientation of the instrument is aligned with the predetermined target plane, and based on the determined alignment, display augmented reality information comprising an indication representing that the current orientation of the instrument is aligned with the predetermined target plane. In some embodiments, the controller is further configured to display the indication of the predetermined target plane for the cut on the bone as a plurality of first indicators, to display the indication of the cut that would be produced by the current orientation of the instrument as a plurality of second indicators, and to display the indication representing that the current orientation of the instrument is aligned with the predetermined target plane as a plurality of third indicators.

### BRIEF DESCRIPTION OF THE DRAWINGS

FIG. 1 depicts a computer aided surgery (CAS) system employing Augmented Reality (AR).

FIG. 2 depicts a schematic of a view displayed on an AR display having virtual indicators overlaid over a real world scene, the indicators including a current orientation of an instrument, a predetermined (e.g., planned) plane for the instrument, and a point where the instrument is successfully aligned with the plane.

FIG. 3 depicts a schematic of a view displayed on an AR display similar to FIG. 2, but with an additional indicator a representing a plane.

FIG. 4 depicts a schematic of a view displayed on an AR display according to another embodiment.

FIG. 5 depicts a schematic of a view displayed on an AR display according to yet another embodiment.

FIG. 6 depicts a schematic of a view displayed on an AR display similar to FIG. 5, but with an additional indicator a representing whether the current orientation is above (+) or below (−) the planned target plane.

FIG. 7 depicts a schematic of a view displayed on an AR display according to yet another embodiment.

FIG. 8 depicts a schematic of a view displayed on an AR display according to yet another embodiment.

FIG. 9 depicts a schematic of a view displayed on an AR display having indicators overlaid over a real world scene, the indicators including a predetermined (e.g., planned) alignment for an instrument and a current adjustment of the instrument to successfully align the instrument.

FIG. 10A depicts a schematic of a view displayed on an AR display according to yet another embodiment, the indicators including a current orientation of an instrument contour and a predetermined (e.g., planned) cut contour for the instrument.

FIG. 10B depicts a schematic of the view displayed on the AR display of FIG. 10A after successful alignment.

### DETAILED DESCRIPTION

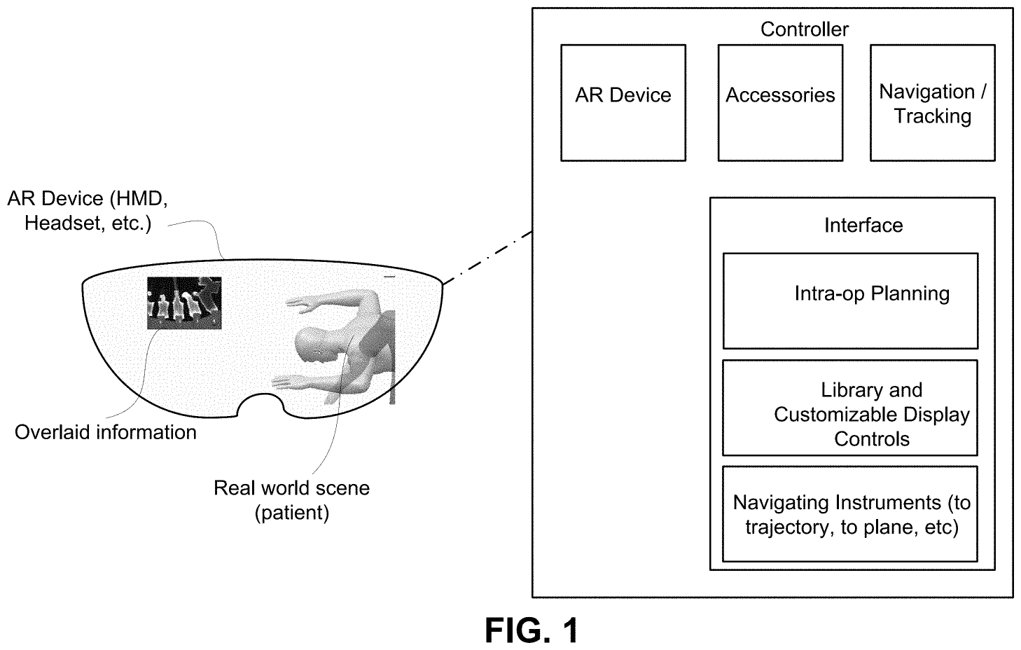

FIG. 1 depicts a computer aided surgery (CAS) system employing Augmented Reality (AR). A user (e.g., surgeon) views a patient or other real-world object (instruments, operating room (OR) features, etc.) while receiving an overlay of virtual information from the controller. The information may be stored information or streamed information. Examples of information include pictures, video, text, warnings, models, simulations, etc. The information displayed may be selectable, pertinent, and customizable. For example, intra-op planning may greatly benefit from AR systems, provided it does not negatively impact workflow. Furthermore, specific use cases, such as position-finding of instruments relative to a patient, may present challenges that may be at least ameliorated by properly configured AR systems.

Methods and implementations are provided to assist a surgeon to perform intra-operative (intra-op) visualization and/or planning from an AR headset, with minimal impact to their workflow. AR provides control to the surgeon, for example, for orthopedic procedures. Example applications include hip surgery (e.g., hip arthroplasty), shoulder surgery, spine surgery, and other orthopedic surgeries. In some embodiments, the system may enhance what the surgeon may see and help the surgeon visualize what they can't see. The display may include virtual targets on the anatomy and information related to the instrument relative to the target. Provided is an AR system that has a user interface (e.g., with a controller) and a display, such as is typically associated with a headset, head-mounted display (HMD), Google Glass, etc. As will be described, navigation/tracking may be provided. In some embodiments, this application is directed to computer aided surgery (CAS) comprising an augmented reality (AR) system configured to display augmented reality information, a position tracking system configured to track positions of objects, an instrument coupled to a navigational tracker detectable by the position tracking system, and a controller configured to determine a position of the instrument, based on the determined position, display augmented reality information using the AR system. The controller may be used to send and receive information to and from the AR system. The controller typically includes a power supply, AC/DC converters, control system interface circuits, and other components included in computer aided surgery. The controller is also configured to perform the systems and methods described herein.

The controller may be configured for navigation and/or tracking. A position tracking system may comprise a tracker comprising a navigation array including a plurality of markers in a unique constellation or geometric arrangement. For example, optical navigation or tracking systems may utilize stereoscopic sensors (of a tracking unit) to detect light emitting diodes (LEDs) or infra-red (IR) light reflected or emitted from one or more optical markers affixed to the array. For example, when the markers are reflective elements, once detected by stereoscopic sensors, the relative arrangement of the elements in the sensors' field of view, in combination with the known geometric arrangement of the elements, may allow the system to determine a three-dimensional position of the array, and hence the instrument. Other examples of tracking systems in include ultrasonic sensors, radio-frequency identification (RFID) sensors or other radio frequency (RF) tracking systems, electromagnetic interference (EMI) tracking systems, etc. The tracking unit may detect the relative motions between any and all trackers in real time.

In another embodiment, the tracker is one that is detectable by a camera of the headset (AR system), or alternatively by a separate camera mounted to the headset, or alternatively by a camera separate and located remotely from the headset. For example, the tracker may be a chest type tracker (e.g., having one or more markers used for camera pose estimation). The tracker may reveal a position of the instrument (e.g., the tracker may help provide complete positioning information of the instrument which may be used by the controller). The camera may capture a position of the tracker. The relative pose or three-dimensional position (e.g., location and orientation) of the tracker may be tracked and shared with the controller. This information may thus identify a location of the instrument to which the tracker is coupled in three-dimensional space given the known and precise relationship between the tracker and the instrument. For example, the controller may be configured to identify a 3D position of a portion of the instrument.

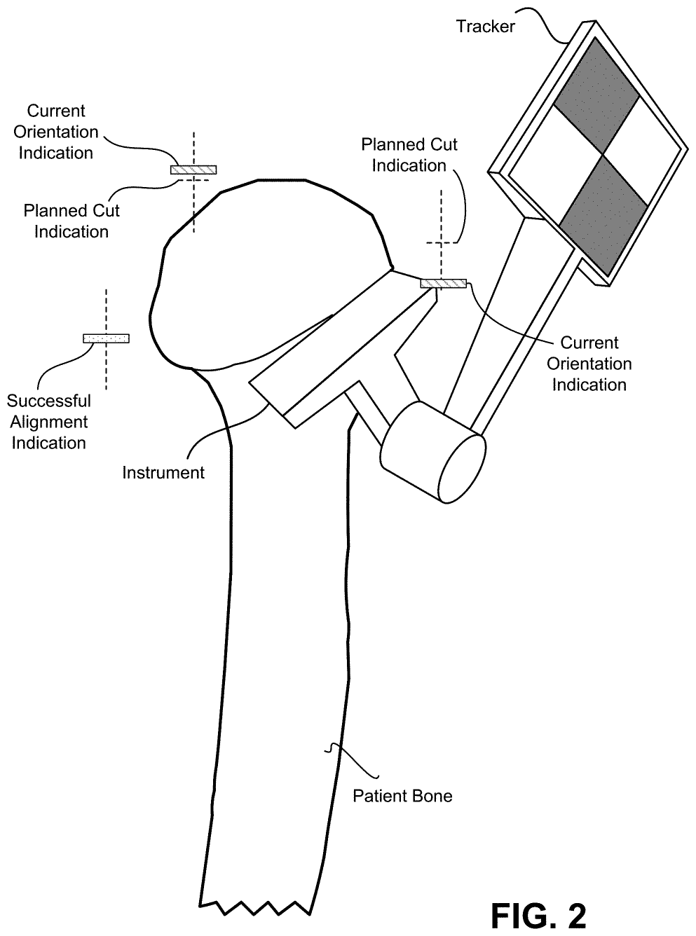

One or more trackers may be attached to a patient, and another tracker attached to the instrument, thus allowing the position of the instrument to be relative to the patient. Although a similar tracker is illustrated in the following FIGS. 2-10B, the system would also work with other trackers (as described above).

The controller may determine (or be informed of) a position of a patient anatomy (a femur, a humerus, a vertebra, etc.). The additional tracker(s) may be present elsewhere in an operating theater, e.g., such as coupled to a surgical table. The additional tracker(s) may assist with tracking an anatomy (e.g., a bone) of interest. A patient coordinate system may be defined to refer to the position of the patient with respect to the instrument.

The tracking unit (e.g., a stereoscopic reflector detecting camera, an AR system camera, or a stand-alone camera) may track the trackers for purposes of determining their relative locations and orientations (e.g., position). A surgeon may view the patient through the AR system and may manipulate the instrument. In some embodiments, a computer aided surgery (CAS) system may comprise an augmented reality (AR) system configured to display augmented reality information, a position tracking system configured to track positions of objects, an instrument coupled to a tracker detectable by the position tracking system, and a controller. The controller may be configured to cause the AR system to display, such as on the headset, augmented reality information about a position of the instrument and a position of the patient. Other augmented reality information may be overlaid (e.g., displayed on the headset as superimposed on the real-world scene). The controller may be configured to, if the instrument moves to another position, cause the AR system to display updated augmented reality information, as will now be described in greater detail. As can be appreciated, using a two-dimensional display to guide a surgeon trying to manipulate or adjust an instrument in three dimensions can be an imposing challenge.

FIG. 2 depicts a schematic of a view displayed on an AR display having virtual indicators overlaid over a real world scene, the indicators including a current orientation of an instrument, a predetermined (e.g., planned) plane for the instrument, and a point where the instrument is successfully aligned with the plane. A surgeon may be looking through an AR device at a patient bone. The bone may be obscured by tissue, and so optionally, an outline of the bone may be displayed (e.g., the controller may be configured to display augmented reality information using the AR system, the augmented reality information comprising an outline of the bone).

The surgeon may have input the type of procedure into the controller. For example, the procedure may be a so-called image-based procedure, where a target plane for a procedure on a bone is planned using three dimensional (3D) images of the bone, for example, computed tomography (CT) data, magnetic resonance imaging (MRI) data, or X-ray data. A position of the bone may be determined (e.g., with one or more trackers (not depicted) attached to the patient). The controller may determine (or be informed of) a position of a patient anatomy (e.g., bone).

A contour of the bone may be determined. The contour may be a periphery of the bone along a plane which intersects the bone. For example, the controller may receive the predetermined (e.g., planned) target plane. The controller may construct a pre-operative (′preop”) bone model. The controller may compute the contour on the bone as the intersection between the preop bone model and the predetermined (e.g., planned) target plane. For example, the contour may be dependent on a shape of the bone and the predetermined (e.g., planned) target plane for cutting the bone (by an instrument or a cut guided by an instrument).

A predetermined (e.g., planned) target plane (may be defined by at least three loci (e.g., it may take three loci to define a plane). In some embodiments, the three loci are spread out relatively equidistantly.

In the case where the instrument is a cut guide, a predetermined (e.g., planned) target plane for the instrument is equivalent to a predetermined (e.g., planned) target plane for a cut on the bone. The augmented reality information may comprise an indication of a predetermined target plane for the cut on the bone. The indication of the predetermined target plane may comprise a plurality of indicators (e.g., as illustrated in FIG. 2, two horizontal dashed line indicators cooperating to define a “planned cut indication”). For clarity, three loci define the plane, but as will be described, the planes are already aligned at one locus (as depicted). Other indicators are contemplated, such as shapes (symmetrical or non-symmetrical) which are filled, cross-hatched, etc., or not. Preferably, all indicators belonging to a particular indication are a same color.

Continuing with the example where the instrument is a cut guide, the controller may be further configured to determine a cut on the bone that would be produced by a current orientation of the instrument. In some embodiments, the cut on the bone that would be produced by a current orientation of the instrument is a planar cut, and thus defines a second contour.

A position of the instrument may be determined using an affixed tracker. Although a chest type tracker is depicted, other trackers, such as an ArUco-type tracker or reflective optical markers are also contemplated. The controller may be configured to use the position of the instrument and predetermined information about the instrument to determine a cut on the bone that would be produced by a current orientation of the instrument. Stated differently, because the cut would be adjacent to the instrument, the instrument defines a currently projected cut plane according to its orientation. It is understood that another instrument might be required to actually make the cut (for example, if the instrument is a cut guide, a saw blade and a handpiece would be needed).

The controller may be configured to display augmented reality information using the AR system, the augmented reality information comprising an indication of a cut that would be produced by a current orientation of the instrument. A current cut plane may be defined by at least three loci (e.g., it may take three loci to define a plane). In some embodiments, the three loci are spread out relatively equidistantly.

The indication of the cut that would be produced by the current orientation of the instrument may comprise a plurality of indicators (e.g., as illustrated in FIG. 2, two horizontal rectangular indicators cooperating to define a “current orientation indication”). Other indicators are contemplated, such as lines or other shapes (symmetrical or non-symmetrical) which are filled, cross-hatched, etc., or not. Preferably, all indicators belonging to a particular indication are a same color which is not the color of another indication, for example, to let a surgeon quickly visualize a particular plane. Preferably, the controller displays the indication of the cut that would be produced by the current orientation of the instrument and the indication of the predetermined target plane in a manner that they will overlap when correctly aligned. For example, an indicator (e.g., of the indication of the cut that would be produced by the current orientation of the instrument) may be associated with an indicator (e.g., of the indication of the predetermined target plane).

As the surgeon moves the instrument, attempting to align the indication of the cut that would be produced by the current orientation of the instrument with indication of the predetermined target plane, the controller determines the new position of the instrument and determines (and displays) an updated indication of the cut that would be produced by the current orientation of the instrument. The controller may be further configured to graphically represent whether the current orientation of the instrument plane is above, below, or at angle to the predetermined target plane. In such case, at each locus, a point on the instrument plane will be above or below the predetermined target plane (unless they are aligned). Accordingly, an angular difference of the instrument plane to the predetermined target plane may be perceived when observing the loci (skew, etc.), by a point on the instrument plane being above or below the predetermined target plane (or aligned).

It is understood that the controller may be further configured to display at least a relative distance between the current orientation of the instrument and the predetermined target plane, e.g., to aid the surgeon in visualizing an amount to adjust a position of the instrument.

Eventually, the surgeon will have moved the instrument so that the current orientation of the instrument is aligned with the predetermined target plane, at least at one locus. The controller may determine alignment (e.g., with a precision sufficient for the procedure). Based on the determined alignment, the controller may be further configured to display augmented reality information comprising an indicator representing that the current orientation of the instrument is aligned with the predetermined target plane (e.g., displayed on the AR display superimposed on the real-world patient). The alignment indication may comprise a plurality of indicators.

In FIG. 2, only a single horizontal rectangular indicator is displayed as part of the “successful alignment indication” as the illustration is meant to depict an intermediate step of the procedure. Ultimately, the surgeon will align the planes at all three loci (for example, one at a time until the planes align) and the successful alignment indication will comprise three successful alignment indicators at the positions formerly occupied by the planned cut indicators. In some embodiments, the alignment indicator replaces the planned target indicator. In some embodiments, the alignment indicator is in a different location. In some embodiments, the alignment indicator is superimposed on another indicator (for example, a flashing planned target indicator, a check mark on top of the planned target indicator, a ring drawn around planned target indicator, etc.). Other successful alignment indicators are contemplated, such as lines or other shapes (symmetrical or non-symmetrical) which are filled, cross-hatched, etc., or not. Preferably, all indicators belonging to a particular indication are a same color which is not the color of another indication, for example, to let a surgeon quickly visualize a particular plane.

The surgeon may pin the instrument in place once the current orientation of the instrument is aligned with the predetermined target plane.

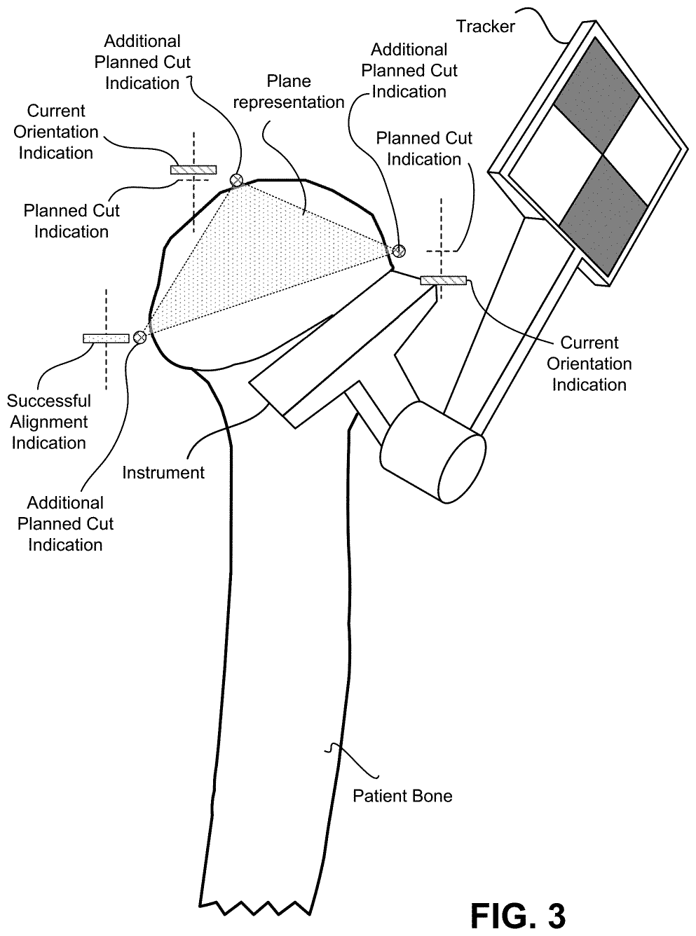

FIG. 3 depicts a schematic of a view displayed on an AR display similar to FIG. 2, and thus the accompanying description of FIG. 2 is equally applicable to FIG. 3 and need not be repeated. However, in FIG. 3, the controller may be further configured to display augmented reality information using the AR system, the augmented reality information comprising an additional indication of the predetermined target plane for the cut on the bone as a plane (denoted as an “additional planned cut indication”). This may aid a surgeon in visualizing a final (e.g., aligned) position of the instrument.

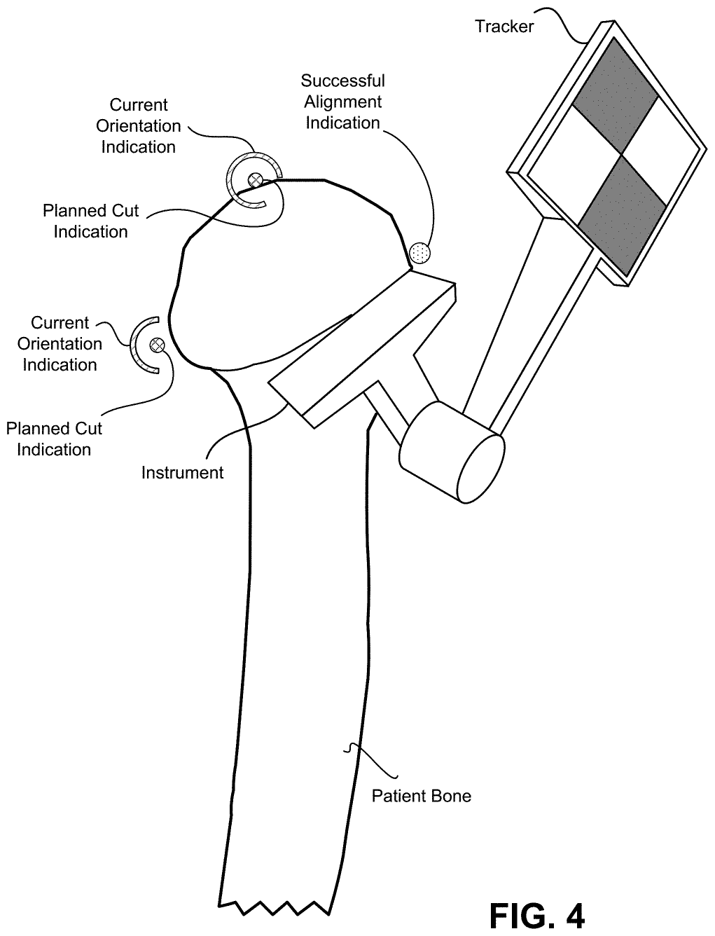

FIG. 4 depicts a schematic of a view displayed on an AR display according to another embodiment. It will be understood that the surgical planning and use aspects are substantially similar to that previously described with respect to FIG. 2, and so this description focuses on the virtual indications displayed on the AR system.

Continuing with the example where the instrument is a cut guide, the controller may be further configured to display augmented reality information that may comprise an indication of a predetermined target plane for the cut on the bone. The indication of the predetermined target plane may comprise a plurality of indicators (e.g., as illustrated in FIG. 4, two filled circular indicators cooperating to define a “planned cut indication”). Other indicators are contemplated, such as lines or shapes (symmetrical or non-symmetrical) which are filled, cross-hatched, etc., or not. Preferably, all indicators belonging to a particular indication are a same color.

The indication of the cut that would be produced by the current orientation of the instrument may comprise a plurality of indicators (e.g., as illustrated in FIG. 4, two semi-circular indicators cooperating to define a “current orientation indication”). Other indicators are contemplated, such as lines or other shapes (symmetrical or non-symmetrical) which are filled, cross-hatched, etc., or not. Preferably, all indicators belonging to a particular indication are a same color which is not the color of another indication, for example, to let a surgeon quickly visualize a particular plane. Preferably, the controller displays the indication of the cut that would be produced by the current orientation of the instrument and the indication of the predetermined target plane in a manner that they will overlap when correctly aligned. For example, an indicator of the indication of the cut that would be produced by the current orientation of the instrument may be associated with an indicator of the indication of the of the predetermined target plane.

As the surgeon moves the instrument, attempting to align the indication of the cut that would be produced by the current orientation of the instrument with indication of the predetermined target plane, the controller determines the new position of the instrument and determines (and displays) an updated indication of the cut that would be produced by the current orientation of the instrument. It is understood that the controller may be further configured to display at least a relative difference between the current orientation of the instrument and the predetermined target plane, e.g., to aid the surgeon in visualizing an amount to adjust a position of the instrument. For example, a more complete semi-circle may indicate that an indicator of the indication of the cut that would be produced by the current orientation of the instrument is closer to alignment with an associated indicator of the indication of the predetermined target plane.

Eventually, the surgeon will have moved the instrument so that the current orientation of the instrument is aligned with the predetermined target plane, at least at one locus. The controller may determine alignment (e.g., with a precision sufficient for the procedure). Based on the determined alignment, the controller may be further configured to display augmented reality information comprising an indicator representing that the current orientation of the instrument is aligned with the predetermined target plane (e.g., displayed on the AR display superimposed on the real-world patient). The alignment indication may comprise a plurality of indicators. In FIG. 4, only a single filled circle indicator is displayed as part of the “successful alignment indication” as the illustration is meant to depict an intermediate step of the procedure. In some embodiments, the alignment indicator is a different size from the planned target indicator.

Ultimately, the surgeon will align the planes at all three loci (for example, one at a time until the planes align) and the successful alignment indication will comprise three successful alignment indicators at the positions formerly occupied by the planned cut indicators. In some embodiments, the alignment indicator replaces the planned target indicator. In some embodiments, the alignment indicator is in a different location. In some embodiments, the alignment indicator is superimposed on another indicator (for example, a flashing planned target indicator, a check mark on top of the planned target indicator, a ring drawn around planned target indicator, etc.). Other successful alignment indicators are contemplated, such as lines or other shapes (symmetrical or non-symmetrical) which are filled, cross-hatched, etc., or not. Preferably, all indicators belonging to a particular indication are a same color which is not the color of another indication, for example, to let a surgeon quickly visualize a particular plane. In some embodiments, all indicators belonging to a particular indication are a same size which is not the size of another indication.

The surgeon may pin the instrument in place once the current orientation of the instrument is aligned with the predetermined target plane.

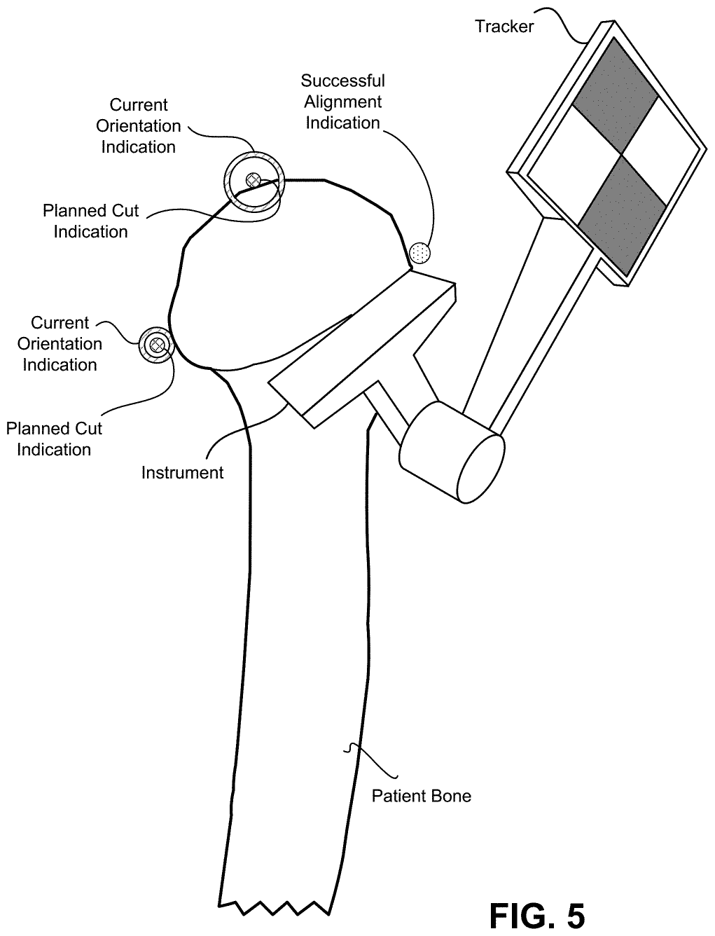

FIG. 5 depicts a schematic of a view displayed on an AR display according to yet another embodiment. It will be understood that the surgical planning and use aspects are substantially similar to that previously described with respect to FIG. 2, and so this description focuses on the virtual indications displayed on the AR system.

Continuing with the example where the instrument is a cut guide, the controller may be further configured to display augmented reality information that may comprise an indication of a predetermined target plane for the cut on the bone. The augmented reality information may comprise an indication of a predetermined target plane for the cut on the bone. The indication of the predetermined target plane may comprise a plurality of indicators (e.g., as illustrated in FIG. 5, two filled circular indicators cooperating to define a “planned cut indication”). Other indicators are contemplated, such as lines or shapes (symmetrical or non-symmetrical) which are filled, cross-hatched, etc., or not. Preferably, all indicators belonging to a particular indication are a same color.

The indication of the cut that would be produced by the current orientation of the instrument may comprise a plurality of indicators (e.g., as illustrated in FIG. 5, two annular (e.g., ring) indicators cooperating to define a “current orientation indication”). Other indicators are contemplated, such as lines or other shapes (symmetrical or non-symmetrical) which are filled, cross-hatched, etc., or not. Preferably, all indicators belonging to a particular indication are a same color which is not the color of another indication, for example, to let a surgeon quickly visualize a particular plane. Preferably, the controller displays the indication of the cut that would be produced by the current orientation of the instrument and the indication of the predetermined target plane in a manner that they will overlap when correctly aligned. For example, an indicator of the indication of the cut that would be produced by the current orientation of the instrument may be associated with an indicator of the indication of the of the predetermined target plane.

As the surgeon moves the instrument, attempting to align the indication of the cut that would be produced by the current orientation of the instrument with indication of the predetermined target plane, the controller determines the new position of the instrument and determines (and displays) an updated indication of the cut that would be produced by the current orientation of the instrument. It is understood that the controller may be further configured to display at least a relative difference between the current orientation of the instrument and the predetermined target plane, e.g., to aid the surgeon in visualizing an amount to adjust a position of the instrument. For example, a smaller ring may indicate that that an indicator of the indication of the cut that would be produced by the current orientation of the instrument is closer to alignment with an associated indicator of the indication of the predetermined target plane.

Eventually, the surgeon will have moved the instrument so that the current orientation of the instrument is aligned with the predetermined target plane, at least at one locus. The controller may determine alignment (e.g., with a precision sufficient for the procedure). Based on the determined alignment, the controller may be further configured to display augmented reality information comprising an indicator representing that the current orientation of the instrument is aligned with the predetermined target plane (e.g., displayed on the AR display superimposed on the real-world patient). The alignment indication may comprise a plurality of indicators. In FIG. 5, only a single filled circle indicator is displayed as part of the “successful alignment indication” as the illustration is meant to depict an intermediate step of the procedure. In some embodiments, the alignment indicator is a different size from the planned target indicator.

Ultimately, the surgeon will align the planes at all three loci (for example, one at a time until the planes align) and the successful alignment indication will comprise three successful alignment indicators at the positions formerly occupied by the planned cut indicators. In some embodiments, the alignment indicator replaces the planned target indicator. In some embodiments, the alignment indicator is in a different location. In some embodiments, the alignment indicator is superimposed on another indicator (for example, a flashing planned target indicator, a check mark on top of the planned target indicator, a ring drawn around planned target indicator, etc.). Other successful alignment indicators are contemplated, such as lines or other shapes (symmetrical or non-symmetrical) which are filled, cross-hatched, etc., or not. Preferably, all indicators belonging to a particular indication are a same color which is not the color of another indication, for example, to let a surgeon quickly visualize a particular plane. In some embodiments, all indicators belonging to a particular indication are a same size which is not the size of another indication.

The surgeon may pin the instrument in place once the current orientation of the instrument is aligned with the predetermined target plane.

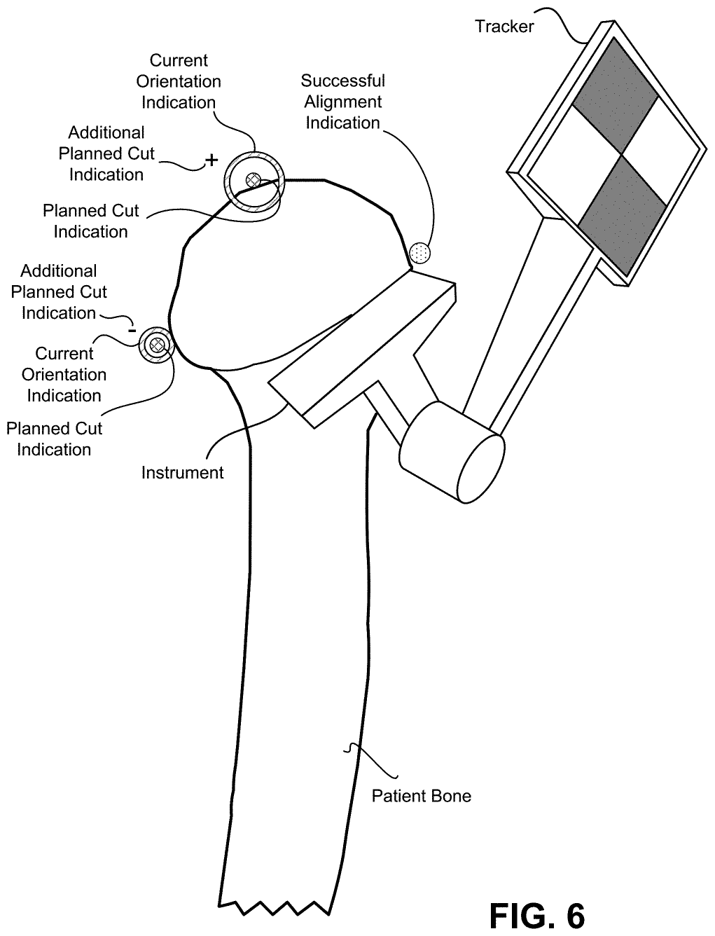

FIG. 6 depicts a schematic of a view displayed on an AR display similar to FIG. 5, and thus the accompanying description of FIG. 5 is equally applicable to FIG. 6 and need not be repeated. However, in FIG. 6, the controller may be further configured to graphically represent whether the current orientation of the instrument is above or below the predetermined target plane. For example, the controller may be further configured to display augmented reality information using the AR system, the augmented reality information comprising an additional indication of the predetermined target plane for the cut on the bone representing whether the current orientation is above (+) or below (−) the planned target plane (denoted as an “additional planned cut indication”). This may aid a surgeon in visualizing a final (e.g., aligned) position of the instrument.

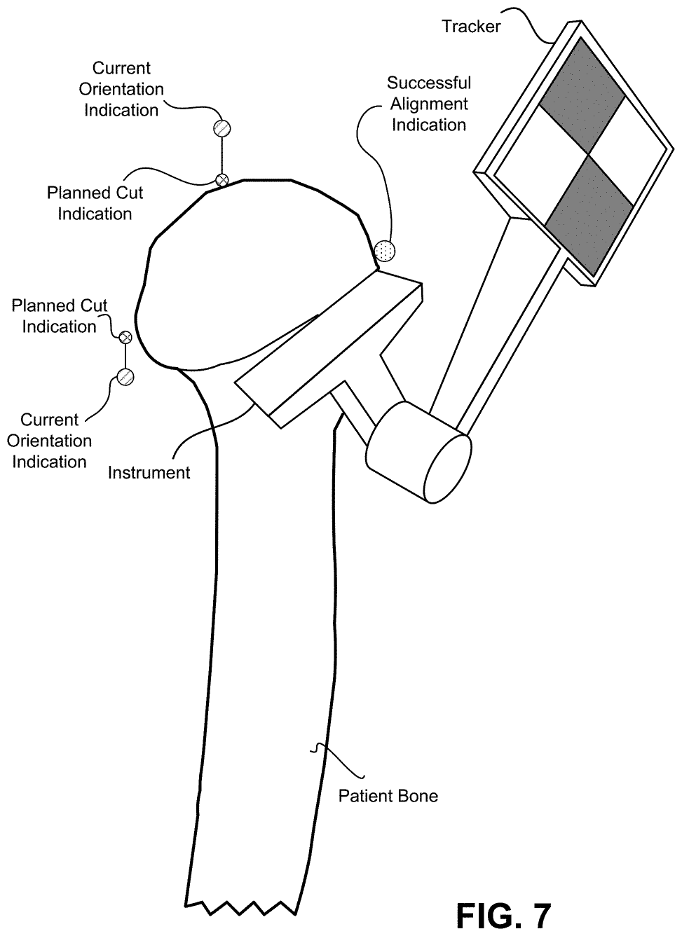

FIG. 7 depicts a schematic of a view displayed on an AR display according to yet another embodiment. It will be understood that the surgical planning and use aspects are substantially similar to that previously described with respect to FIG. 2, and so this description focuses on the virtual indications displayed on the AR system.

Continuing with the example where the instrument is a cut guide, the controller may be further configured to display augmented reality information that may comprise an indication of a predetermined target plane for the cut on the bone. The augmented reality information may comprise an indication of a predetermined target plane for the cut on the bone. The indication of the predetermined target plane may comprise a plurality of indicators (e.g., as illustrated in FIG. 7, two filled circular indicators cooperating to define a “planned cut indication”). Other indicators are contemplated, such as lines or shapes (symmetrical or non-symmetrical) which are filled, cross-hatched, etc., or not. Preferably, all indicators belonging to a particular indication are a same color.

The indication of the cut that would be produced by the current orientation of the instrument may comprise a plurality of indicators (e.g., as illustrated in FIG. 7, two relatively larger filled circular indicators cooperating to define a “current orientation indication”). Other indicators are contemplated, such as lines or other shapes (symmetrical or non-symmetrical) which are filled, cross-hatched, etc., or not. Preferably, all indicators belonging to a particular indication are a same color which is not the color of another indication, for example, to let a surgeon quickly visualize a particular plane. In some embodiments, the current orientation indicator is a different size from the planned target indicator.

Preferably, the controller displays the indication of the cut that would be produced by the current orientation of the instrument and the indication of the predetermined target plane in a manner that they will overlap when correctly aligned. For example, an indicator of the indication of the cut that would be produced by the current orientation of the instrument may be associated with an indicator of the indication of the of the predetermined target plane.

As the surgeon moves the instrument, attempting to align the indication of the cut that would be produced by the current orientation of the instrument with indication of the predetermined target plane, the controller determines the new position of the instrument and determines (and displays) an updated indication of the cut that would be produced by the current orientation of the instrument. The controller may be further configured to graphically represent whether the current orientation of the instrument is above or below the predetermined target plane. It is understood that the controller may be further configured to display at least a relative distance between the current orientation of the instrument and the predetermined target plane, e.g., to aid the surgeon in visualizing an amount to adjust a position of the instrument.

Eventually, the surgeon will have moved the instrument so that the current orientation of the instrument is aligned with the predetermined target plane, at least at one locus. The controller may determine alignment (e.g., with a precision sufficient for the procedure). Based on the determined alignment, the controller may be further configured to display augmented reality information comprising an indicator representing that the current orientation of the instrument is aligned with the predetermined target plane (e.g., displayed on the AR display superimposed on the real-world patient). The alignment indication may comprise a plurality of indicators. In FIG. 7, only a single relatively largest filled circle indicator is displayed as part of the “successful alignment indication” as the illustration is meant to depict an intermediate step of the procedure. In some embodiments, the alignment indicator is a different size from both the planned target indicator and the current orientation indicator.

Ultimately, the surgeon will align the planes at all three loci (for example, one at a time until the planes align) and the successful alignment indication will comprise three successful alignment indicators at the positions formerly occupied by the planned cut indicators. In some embodiments, the alignment indicator replaces the planned target indicator. In some embodiments, the alignment indicator is in a different location. In some embodiments, the alignment indicator is superimposed on another indicator (for example, a flashing planned target indicator, a check mark on top of the planned target indicator, a ring drawn around planned target indicator, etc.). Other successful alignment indicators are contemplated, such as lines or other shapes (symmetrical or non-symmetrical) which are filled, cross-hatched, etc., or not. Preferably, all indicators belonging to a particular indication are a same color which is not the color of another indication, for example, to let a surgeon quickly visualize a particular plane. In some embodiments, all indicators belonging to a particular indication are a same size which is not the size of another indication.

The surgeon may pin the instrument in place once the current orientation of the instrument is aligned with the predetermined target plane.

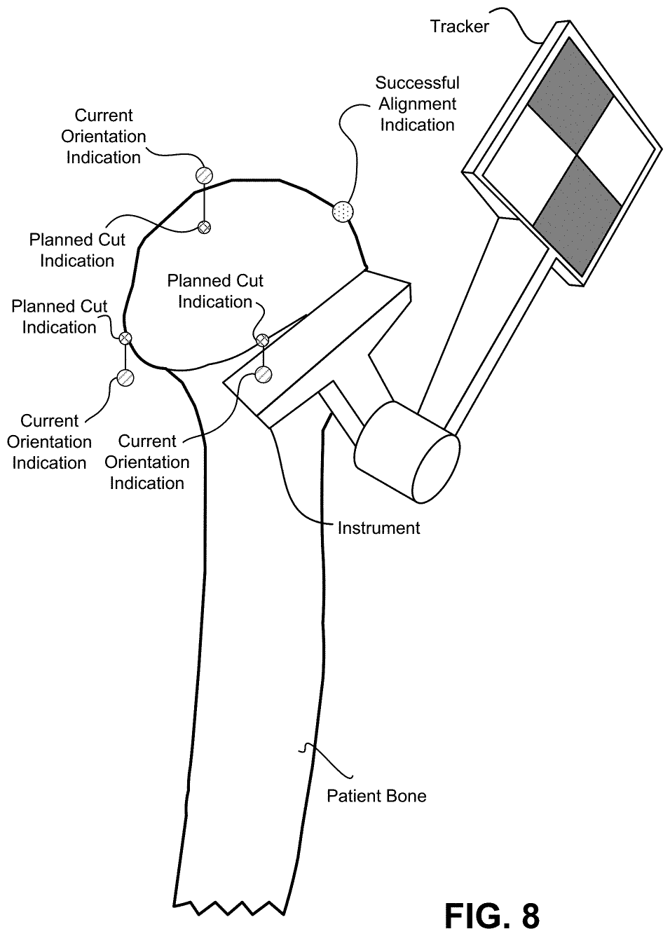

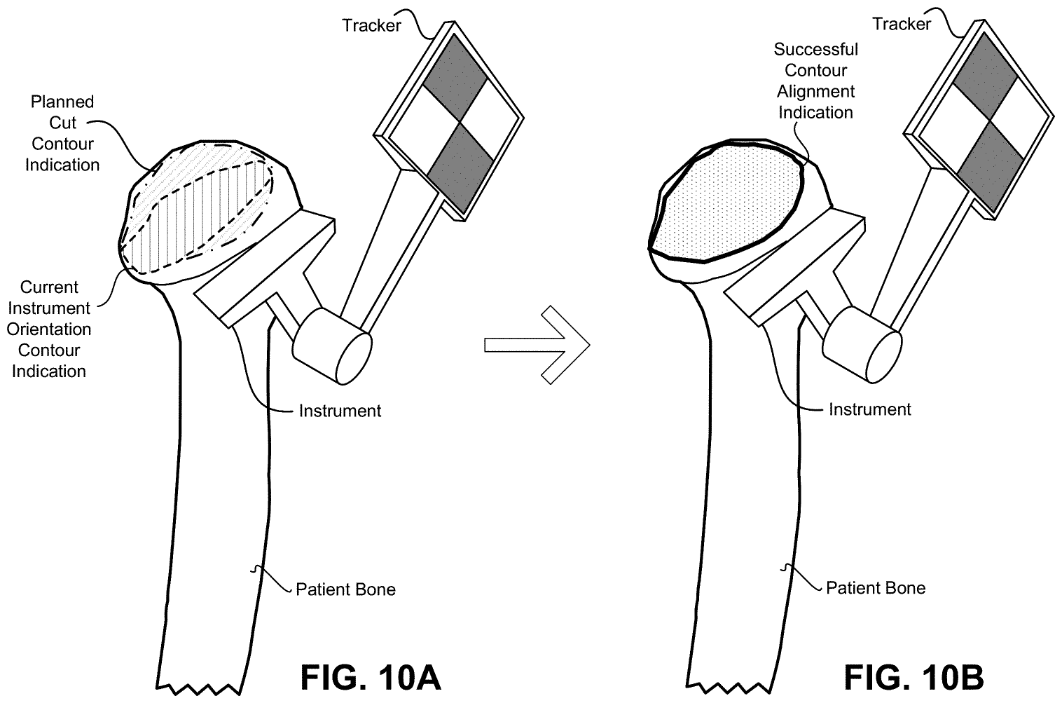

FIG. 8 depicts a schematic of a view displayed on an AR display similar to FIG. 7, and thus the accompanying description of FIG. 7 is equally applicable to FIG. 8 and need not be repeated. However, in FIG. 8, the controller may be further configured to determine planes at four loci. Accordingly, the controller may be further configured to display augmented reality information using the AR system, the augmented reality information comprising four pairs of indicators, each pair comprising an indicator of the indication of the cut that would be produced by the current orientation of the instrument and an associated indicator of the indication of the of the predetermined target plane. Ultimately, the surgeon will align the planes at all four loci (for example, one at a time until the planes align) and the successful alignment indication will comprise four successful alignment indicators at the positions formerly occupied by the planned cut indicators. Other numbers of loci are contemplated up to and including a number of loci sufficient to form a line (see FIGS. 10A-B).

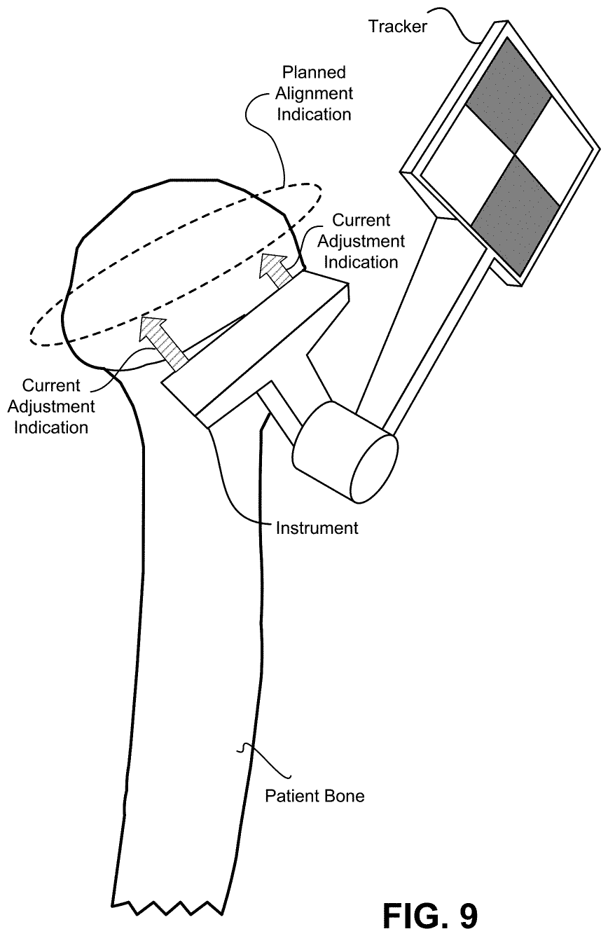

FIG. 9 depicts a schematic of a view displayed on an AR display having indicators overlaid over a real world scene, the indicators including a predetermined (e.g., planned) alignment for an instrument and a current adjustment of the instrument to successfully align the instrument. In this embodiment, an initial alignment for the instrument is provided. A surgeon may be looking through an AR device at a patient bone. The controller may determine (or be informed of) a position of a patient anatomy (e.g., bone). The surgeon may have input the type of procedure into the controller. For example, the procedure may require initial placement of a tracked cut guide instrument. Optionally, an outline of the bone may be displayed (e.g., the controller may be configured to display augmented reality information using the AR system, the augmented reality information comprising an outline of the bone).

A predetermined (e.g., planned) alignment for the instrument may be determined (e.g., by the controller), for example, a position where the instrument should abut the bone. The augmented reality information may comprise an indication of a planned alignment. A planned alignment indication may be displayed, such as lines or shapes (symmetrical or non-symmetrical) which are filled, cross-hatched, etc., or not. Preferably, all indicators belonging to a particular indication are a same color.

A position of the instrument may be determined using the affixed tracker. The controller may be configured to determine a current adjustment to reach the planned alignment. The controller may display augmented reality information using the AR system, the augmented reality information comprising a current adjustment indication (e.g., to reach the planned alignment). As illustrated in FIG. 9, two arrow indicators cooperate to define a “current orientation indication”). Other indicators are contemplated, such as lines or other shapes (symmetrical or non-symmetrical) which are filled, cross-hatched, etc., or not. Preferably, all indicators belonging to a particular indication are a same color which is not the color of another indication, for example, to let a surgeon quickly visualize a particular plane. In some embodiments, the controller may be further configured to display at least a relative difference between the current adjustment and the planned, e.g., to aid the surgeon in visualizing an amount to adjust a position of the instrument.

In some embodiments, current orientation indication relates to a projection or indication for spike or pin placement (e.g., of a cut guide). This may be useful for showing where they intersect (or don't intersect) with the bone surface. A surgeon could move the guide to avoid tissue damage in a tissue sparing approach and optimize pin location in the bone. The optimized location may be a planned alignment.

As the surgeon moves the instrument, attempting to align the indications, the controller determines the new position of the instrument and determines (and displays) an updated current adjustment indication. Eventually, the surgeon will have moved the instrument so that the instrument reaches the planned alignment. The controller may determine alignment (e.g., with a precision sufficient for the procedure). Based on the determined alignment, the controller may be further configured to display augmented reality information comprising an indicator representing that the instrument is successfully aligned with the planned alignment (e.g., displayed on the AR display superimposed on the real-world patient).

FIG. 10A depicts a schematic of a view displayed on an AR display according to yet another embodiment. It will be understood that the surgical planning and use aspects are substantially similar to that previously described with respect to FIG. 2, and so this description focuses on the virtual indications displayed on the AR system.

The augmented reality information may comprise a representation of a contour based on a predetermined target plane for the cut on the bone (e.g., as illustrated in FIG. 10A, a “planned cut contour indication”). The indication may be dashed or solid, and filled, cross-hatched, etc., or not. Preferably, the indication is a first color.

The augmented reality information may comprise a representation of a contour based on a cut on the bone that would be produced by a current orientation of the instrument (e.g., as illustrated in FIG. 10A, a “current instrument orientation contour indication”). The indication may be dashed or solid, and filled, cross-hatched, etc., or not. Preferably, the indication is a second color (e.g., different from the first color of the planned cut contour indication), for example, to let a surgeon quickly visualize a particular contour.

It should be appreciated that planned cut contour indication and the current instrument orientation contour indication will overlap when the instrument is correctly aligned.

As the surgeon moves the instrument, attempting to align the cut contour indication and the current instrument orientation contour indication, the controller determines the new position of the instrument and determines (and displays) an updated current instrument orientation contour indication. The controller is further configured to graphically represent whether the current orientation of the instrument is above or below the predetermined target plane, e.g., to aid the surgeon in visualizing an adjustment to a position of the instrument.

FIG. 10B depicts a schematic of the view displayed on the AR display of FIG. 10A after successful alignment, that is, after the surgeon has moved the instrument so that the planned cut contour indication and the current instrument orientation contour indication are aligned. The controller may determine alignment (e.g., with a precision sufficient for the procedure). Based on the determined alignment, the controller may be further configured to display augmented reality information comprising an indication representing that the current orientation of the instrument is aligned with the predetermined target plane (e.g., displayed on the AR display superimposed on the real-world patient). In some embodiments, the successful contour alignment indication replaces the planned cut contour indication and is a different color (e.g., a third color). In some embodiments, the successful contour alignment indication is in a different location. In some embodiments, the successful contour alignment indication is superimposed on another indication (for example, a flashing planned cut contour indication, a check mark on top of the planned cut contour indication, a ring drawn around planned cut contour indication, etc.). Other successful alignment indications are contemplated, such as lines or other shapes (symmetrical or non-symmetrical) which are filled, cross-hatched, etc., or not. The surgeon may pin the instrument in place once the current orientation of the instrument is aligned with the predetermined target plane.

In a first embodiment, a computer aided surgery (CAS) system is provided. The CAS system comprises an augmented reality (AR) system configured to display augmented reality information; a position tracking system configured to track positions of objects; an instrument coupled to a tracker detectable by the position tracking system; and a controller configured to: display augmented reality information using the AR system, the augmented reality information comprising: an indication of a predetermined target plane for a cut on the bone; and an indication of a cut that would be produced by a current orientation of the instrument; determine, as the instrument is moved, when the cut that would be produced by the current orientation of the instrument is aligned with the predetermined target plane; and based on the determined alignment, display augmented reality information comprising an indication representing that the current orientation of the instrument is aligned with the predetermined target plane. In some embodiments, the controller may be further configured to construct a pre-operative bone model. In some embodiments, the controller may be further configured to compute a contour on the bone as the intersection between the bone model and the predetermined (e.g., planned) target plane. For example, the contour may be dependent on a shape of the bone and the predetermined (e.g., planned) target plane for cutting the bone (by an instrument or a cut guided by an instrument).

In some embodiments, the controller may be further configured to display the indication of the predetermined target plane for the cut on the bone as a plurality of first indicators. In some embodiments, the controller may be further configured to display the indication of the cut that would be produced by the current orientation of the instrument as a plurality of second indicators. In some embodiments, the controller may be further configured to display the second indicators so that a size of the second indicators change as the current orientation of the instrument becomes more closely aligned with the predetermined target plane.

In some embodiments, the controller may be further configured to display at least three first indicators and at least three second indicators.

In some embodiments, the controller may be further configured to display the indication representing that the current orientation of the instrument is aligned with the predetermined target plane as a plurality of third indicators. In some embodiments, the controller may be further configured to display the plurality of first indicators as a first color, the plurality of second indicators as a second color, and the plurality of third indicators as a third color. In some embodiments, the controller may be further configured to display the plurality of first indicators as a first size, the plurality of second indicators as a second size, and the plurality of third indicators as a third size. In some embodiments, the controller may be further configured to replace the plurality of first indicators with the plurality of third indicators.

In some embodiments, the controller may be further configured to display the indication of the predetermined target plane for the cut on the bone as a plane.

In some embodiments, the controller may be further configured to graphically represent whether the current orientation of the instrument is above, below, or at an angle to, the predetermined target plane. In some embodiments, the controller may be further configured to display the indication of the predetermined target plane for the cut on the bone as a plurality of first indicators and to display the indication of the cut that would be produced by the current orientation of the instrument as a plurality of second indicators. In some embodiments, the controller may be further configured to display each second indicator as above or below its respective first indicator depending upon whether the current orientation of the instrument is above or below the predetermined target plane (if they are not aligned). In some embodiments, the controller may be further configured to display a plus adjacent to a second indicator if the current orientation of the instrument is above the predetermined target plane, and a minus adjacent to a second indicator if the current orientation of the instrument is below the predetermined target plane.

In some embodiments, the controller may be further configured to graphically represent a distance between the current orientation of the instrument and the predetermined target plane. In some embodiments, the controller may be further configured to display the indication of the predetermined target plane for the cut on the bone as a plurality of first indicators and to display the indication of the cut that would be produced by the current orientation of the instrument as a plurality of second indicators. In some embodiments, the controller may be further configured to display a second indicator as a greater size than a first indicator, and display the size of the second indicator decreasing as the current orientation of the instrument is brought closer to alignment with the predetermined target plane. In some embodiments, the controller may be further configured to display a second indicator as above or below its respective first indicator, and display the distance decreasing as the current orientation of the instrument is brought closer to alignment with the predetermined target plane.

In a second embodiment, a method of computer aided surgery (CAS) is provided. The method may be a pre-operative planning method. The method comprises determining a position of an instrument, wherein the instrument is coupled to a navigational tracker detectable by a position tracking system; displaying, on an augmented reality (AR) system, augmented reality information comprising an indication of a cut on a bone of a patient that would be produced by a current orientation of the instrument and an indication of a predetermined target plane for the cut; determining, as the instrument is moved, when the current orientation of the instrument is aligned with the predetermined target plane; and based on the determined alignment, displaying augmented reality information comprising an indication representing that the current orientation of the instrument is aligned with the predetermined target plane. In some embodiments, the method further comprises displaying the indication of the predetermined target plane for the cut on the bone as a plurality of first indicators; displaying the indication of the cut that would be produced by the current orientation of the instrument as a plurality of second indicators; and graphically representing at least one of whether the current orientation of the instrument is above or below the predetermined target plane or a distance between the current orientation of the instrument and the predetermined target plane.

The embodiments of the present disclosure described above are intended to be merely examples; numerous variations and modifications within the scope of this disclosure. Accordingly, the disclosure is not to be limited by what has been particularly shown and described. All publications and references cited herein are expressly incorporated by reference in their entirety, except for any definitions, subject matter disclaimers or disavowals, and except to the extent that the incorporated material is inconsistent with the express disclosure herein, in which case the language in this disclosure controls.

1. A computer aided surgery (CAS) system, comprising:
an augmented reality (AR) system configured to display augmented reality information;
a position tracking system configured to track positions of objects;
an instrument coupled to a tracker detectable by the position tracking system; and
a controller configured to:
display augmented reality information using the AR system, the augmented reality information comprising:
an indication of a predetermined target plane for a cut on the bone; and
an indication of a cut that would be produced by a current orientation of the instrument;
determine, as the instrument is moved, when the cut that would be produced by the current orientation of the instrument is aligned with the predetermined target plane; and
based on the determined alignment, display augmented reality information comprising an indication representing that the current orientation of the instrument is aligned with the predetermined target plane.

2. The system of claim 1, wherein the controller is further configured to display the indication of the predetermined target plane for the cut on the bone as a plurality of first indicators.

3. The system of claim 2, wherein the controller is further configured to display the indication of the cut that would be produced by the current orientation of the instrument as a plurality of second indicators.

4. The system of claim 3, wherein there are at least three first indicators and at least three second indicators.

5. The system of claim 3, wherein the controller is further configured to display the indication representing that the current orientation of the instrument is aligned with the predetermined target plane as a plurality of third indicators.

6. The system of claim 5, wherein controller is further configured to display the plurality of first indicators as a first color, the plurality of second indicators as a second color, and the plurality of third indicators as a third color.

7. The system of claim 5, wherein controller is further configured to display the plurality of first indicators as a first size, the plurality of second indicators as a second size, and the plurality of third indicators as a third size.

8. The system of claim 5, wherein controller is further configured to replace the plurality of first indicators with the plurality of third indicators.

9. The system of claim 1, wherein the controller is further configured to display the indication of the predetermined target plane for the cut on the bone as a plane.

10. The system of claim 3, wherein the controller is further configured to display the second indicators so that a size of the second indicators change as the current orientation of the instrument becomes more closely aligned with the predetermined target plane.

11. The system of claim 1, wherein the controller is further configured to graphically represent whether the current orientation of the instrument is above, below, or at an angle to, the predetermined target plane.

12. The system of claim 11, wherein the controller is further configured to display the indication of the predetermined target plane for the cut on the bone as a plurality of first indicators and to display the indication of the cut that would be produced by the current orientation of the instrument as a plurality of second indicators.

13. The system of claim 12, and wherein each second indicator is displayed as above or below its respective first indicator depending upon whether the current orientation of the instrument is above or below the predetermined target plane.

14. The system of claim 12, wherein a plus is displayed adjacent to a second indicator if the current orientation of the instrument is above the predetermined target plane, and a minus is displayed adjacent to a second indicator if the current orientation of the instrument is below the predetermined target plane.

15. The system of claim 1, wherein the controller is further configured to graphically represent a distance between the current orientation of the instrument and the predetermined target plane.

16. The system of claim 15, wherein the controller is further configured to display the indication of the predetermined target plane for the cut on the bone as a plurality of first indicators and to display the indication of the cut that would be produced by the current orientation of the instrument as a plurality of second indicators.

17. The system of claim 16, wherein a second indicator is displayed as a greater size than a first indicator, the size of the second indicator decreasing as the current orientation of the instrument is brought closer to alignment with the predetermined target plane.

18. The system of claim 16, wherein a second indicator is displayed as above or below its respective first indicator, the distance decreasing as the current orientation of the instrument is brought closer to alignment with the predetermined target plane.

19. A method of computer aided surgery (CAS) pre-operative planning, comprising:
determining a position of an instrument, wherein the instrument is coupled to a navigational tracker detectable by a position tracking system;
displaying, on an augmented reality (AR) system, augmented reality information comprising an indication of a cut on a bone of a patient that would be produced by a current orientation of the instrument and an indication of a predetermined target plane for the cut;
determining, as the instrument is moved, when the current orientation of the instrument is aligned with the predetermined target plane; and
based on the determined alignment, displaying augmented reality information comprising an indication representing that the current orientation of the instrument is aligned with the predetermined target plane.

20. The method of claim 19, further comprising:
displaying the indication of the predetermined target plane for the cut on the bone as a plurality of first indicators;
displaying the indication of the cut that would be produced by the current orientation of the instrument as a plurality of second indicators; and
graphically representing at least one of whether the current orientation of the instrument is above or below the predetermined target plane or a distance between the current orientation of the instrument and the predetermined target plane.
